(19) United States
(12) Patent Application Publication
(10) Pub. No.: US 2015/0025019 A1
(43) Pub. Date: Jan. 22, 2015

(54) Method for Dosage Adjustment of Drugs that Prevent Elastic Fiber Breakdown

(71) Applicants: Jerome Owen Cantor, Brooklyn, NY (US); Bronislava Shteyngart, Brooklyn, NY (US); Gerard Turino, New York, NY (US)

(72) Inventors: Jerome Owen Cantor, Brooklyn, NY (US); Bronislava Shteyngart, Brooklyn, NY (US); Gerard Turino, New York, NY (US)

(21) Appl. No.: 14/333,897

(22) Filed: Jul. 17, 2014

Related U.S. Application Data
(60) Provisional application No. 61/847,609, filed on Jul. 18, 2013.

(51) Int. Cl. A61K 31/728; G01N 33/68

(52) U.S. Cl. CPC A61K 31/728; G01N 33/6884
USPC 514/20.4; 514/54

(57) ABSTRACT

Elastic fiber damage is responsible for the distention and rupture of alveolar walls in chronic obstructive pulmonary disease (COPD). The current invention comprises a method of adjusting the a dosage of a drug that prevents such elastic fiber damage by employing a feedback loop consisting of measuring the levels of the unique elastic fiber breakdown products, desmosine and isodesmosine in body fluids, preferably sputum. The recent discovery that sputum levels of desmosine and isodesmosine are responsive to treatment with aerosolized hyaluronan in COPD patients makes dosage adjustment both feasible and desirable. This process of dosage adjustment may be applicable to any drug that prevents lung elastic fiber damage, such as alpha-1-antitrypsin, other anti-elastase agents, and antioxidants. It is anticipated that such dosage adjustment will become the “standard of care” in the treatment of COPD patients.

### RELATED APPLICATIONS

This application claims benefit of provisional patent No. 61/847,609, filed on Jul. 18, 2013.

### BACKGROUND OF THE INVENTION

Chronic obstructive pulmonary disease (COPD) is a complex disease involving damage to both the airways and parenchyma of the lung (1-3). Individuals with COPD may suffer from bronchitis, asthma, pulmonary emphysema, or a combination of these conditions. According to recent estimates, COPD afflicts about 24 million people in the US, with about half of the cases undiagnosed (4).

Although the disease may initially be limited to the upper respiratory tract, a significant number of COPD patients eventually develop pulmonary emphysema. As the disease progresses, imbalances in ventilation and blood perfusion lead to impaired oxygenation of the blood and ultimately, right-sided heart failure.

Currently, there is no effective treatment for pulmonary emphysema. Therapy for the disease is largely palliative, involving administration of oxygen to help alleviate shortness of breath. Nevertheless, the early diagnosis of pulmonary emphysema might permit the institution of certain measures that could mitigate the long-term effects of the disease. Since the disease generally advances over a period of decades, cessation of smoking and administration of anti-inflammatory agents at an early stage might slow the progression of lung injury so that the worst symptoms of the disease may be delayed until extreme old age, effectively eliminating them from the lives of most patients.

However, achieving such a favorable outcome is greatly limited by the fact that there is currently no effective means of detecting incipient pulmonary emphysema. Usually, the presence of the disease is discovered only after there is significant loss of lung volume. At this point, the disease process is well under way and less amenable to treatment.

The availability of a sensitive and specific test for the disease would permit early therapeutic intervention and therefore decrease the possibility of respiratory failure. Since damage to elastic fibers is a basic feature of pulmonary emphysema, the ability to specifically measure the breakdown products of these fibers might provide an important means of identifying those at risk for developing this disease.

Relationship Between Pulmonary Emphysema and Elastic Fiber Breakdown Products

The association between pulmonary emphysema and elastic fiber injury has become increasingly clear over the past four decades. The use of the enzyme, papain, to experimentally induce emphysema represented an initial breakthrough in understanding the pathogenesis of the disease (5). Originally intended as a possible treatment for interstitial pulmonary fibrosis, intratracheal instillment of papain produced prominent pulmonary air-space enlargement, similar to that seen in human emphysema. The finding had added significance because it came at a time when the role of alpha-1-antiproteinase deficiency in this disease was just being understood (6).

Both observations emphasized the importance of proteolysis as a cause of the pulmonary emphysema. An imbalance between lung proteases and their inhibitors was hypothesized to be responsible for the air-space enlargement that characterizes the disease. It was proposed that an excess of elastase activity in the pulmonary parenchyma caused damage to the elastic fiber network of the lung, leading to dilatation and rupture of alveoli, reduced gas-exchange, and eventual respiratory failure.

### Currently Available Methods to Assess COPD

The clinical progression of pulmonary emphysema is currently assessed by measuring the amount of air that a patient can forcibly expel from the lungs following deep inhalation. The forced expiratory volume in one second (FEV1) is a particularly useful parameter for determining the presence of this disease (7). Starting from early adulthood, the FEV1 of a normal individual decreases about 20-30 cc per year, whereas a patient with emphysema loses about 50-150 cc per year. Nevertheless, the usefulness of FEV1 is limited by the fact that such measurements have a significant degree of variability and must therefore be repeated over a period of years to conclusively demonstrate the loss of lung function (8).

A more sensitive measure of lung damage that is currently being developed involves the use of high-resolution computerized tomography (HRCT). This technique provides a visual map of lung density that reflects damage to the alveolar compartment (9-11). However, the usefulness of HRCT is limited by the sensitivity of the imaging process. At present, it is not an effective means of determining the earliest changes of pulmonary emphysema. Likewise, it cannot reliably monitor the rate of lung destruction over short periods of time, and is therefore of limited use in assessing the efficacy of therapeutic intervention.

As a result of the limitations of these testing procedures, a number of biochemical parameters have been proposed as possible markers of the disease process. Various inflammatory mediators are elevated in pulmonary emphysema and may serve to monitor the inflammatory process associated with the disease (12). However, none of these markers are specific for pulmonary emphysema and may therefore reflect the presence of concomitant disease processes in the lung and other organs.

### Elastic Fiber Breakdown Products

Perhaps the most reliable biochemical indicator of lung damage due to pulmonary emphysema is elastic fiber breakdown products. Mature elastic fibers generally do not undergo turnover unless they are broken down by enzymes or other injurious agents. Consequently, the presence of elastic fiber fragments in bodily fluids such as blood, urine, and sputum is evidence that they are being degraded.

Elastic fibers have a highly specialized structure, consisting of an amorphous core elastin protein surrounded by layers of microfibrils (14). The elastin protein is composed of a network of polypeptides joined together by the coalescence of lysine side-chains into crosslinking structures, particularly desmosine and isodesmosine (14). These crosslinking amino acids are specific for the elastin protein and their recovery from the respiratory tract is evidence of elastic fiber degradation.

Since emphysema is related to elastic fiber damage, measurement of the rate of breakdown of these fibers may be extremely useful in assessing the progression of the disease. To date, attempts to measure elastic fiber breakdown products have involved sampling of blood, urine, tracheal aspirates, and bronchoalveolar lavage fluid (15-19). The use of blood or urine is complicated by the fact that both fluids contain elastic fiber breakdown products from sites other than the lung, including elastic fiber-rich tissues such as blood vessels and cartilage. Consequently, diseases such as arteriosclerosis or osteoarthritis may obscure the component of elastic fiber injury due to pulmonary emphysema. The use of tracheal aspirates and bronchoalveolar lavage removes this complication, but the required procedures entail a significant degree of patient discomfort and are therefore unsuitable for repeated measurements.

The only source of elastic fiber breakdown products that is readily accessible and specific for lung injury is sputum. The presence of elastic fibers in the sputum of patients with necrotizing pneumonia is well-documented (20-22). However, the quantitative measurement of elastic fiber breakdown products in sputum has only recently been attempted, and there has been no correlation between the levels of these breakdown products in sputum and degree of lung injury. Nevertheless, the availability of such a test would have multiple applications, including monitoring the therapeutic efficacy of agents used to treat pulmonary emphysema and identification of individuals with incipient disease.

### Measuring Desmosine and Isodesmosine in Sputum

The procedure for measuring desmosine and isodesmosine in sputum was originally described by the author, and involves isolation of these crosslinks by chemical degradation of the sputum into its component amino acids (23). A simple procedure to achieve this end involves hydrolyzing the sample with an acid such as HCl or a base such as NaOH, according to well-established procedures. The total amount of desmosine and isodesmosine in the hydrolysate could then be quantified by a number of available techniques such as radioimmunoassay, electrophoresis, chromatography, or mass spectrometry, and normalized to one or more parameters, such as total protein content (17, 24-26).

Recently, investigators have performed measurements of sputum in both COPD patients and normal individuals to determine the feasibility and reliability of using this type of test (29). A highly sensitive method was used for measuring desmosine and isodesmosine in sputum hydrolysates, involving high-performance liquid chromatography followed by electrospray ionization mass spectrometry.

The results of that study indicate that COPD patients have significantly higher amounts of desmosine and isodesmosine in their sputum compared to normal individuals. Furthermore, the levels associated with the normal group were generally below the detection threshold of the mass spectrometer, suggesting that the presence of even minute amounts of these crosslinks is a harbinger of lung disease.

Subsequently, another laboratory found that the level of desmosine and isodesmosine in sputum from COPD patients with emphysema was higher than that of COPD patients without emphysema, although the difference was not statistically different (30). The fact that COPD patients without emphysema contain measurable amounts of desmosine and isodesmosine suggests that elastic fiber injury occurs prior to the clinical detection of alveolar damage and may spread from the airways to the lung parenchyma as the disease progresses.

Potential Problems Associated with the Use of a Sputum Assay

Prior to clinical application of a sputum assay for desmosine and isodesmosine, both the sensitivity and specificity of the procedure will need to be determined. The fact that fragments of elastic fibers may readily be visualized in the sputum of patients with inflammatory lung diseases suggests that sensitivity should not be a problem in moderate to severe cases of pulmonary emphysema. What is less certain is the ability to detect elastic fiber breakdown products in patients with early disease.

Nevertheless, procedures such as radioimmunoassay or mass spectrometry allow for detection of nanogram quantities of desmosine and isodesmosine (6), and the procurement of ample amounts of sputum from the lower respiratory tract should improve the chances of detecting these amino acids. Standardizing the procedure may require the use of techniques to induce sputum production, thereby assuring recovery of a representative sample from the deeper regions of the respiratory tract (31).

The evaluation of test specificity is more complex since it will require examination of a large number of sputum samples from both patients and normal subjects to determine the range of values. The most important question to be answered is whether individuals with pulmonary emphysema have significantly higher levels of desmosine and isodesmosine than those who have no disease. This information will determine the value of the test as a screening procedure for persons who have a greater than normal risk of developing emphysema.

Another critical question is whether the desmosine and isodesmosine content of sputum samples actually reflects degradative processes at the alveolar level. Since sputum is generally derived from the central airways, there is the possibility that it does not contain elastin breakdown products derived from the alveolar compartment of the lung. The desmosine and isodesmosine in the sputum may only reflect degradative processes in the upper airways or perhaps be contaminated by elastic fiber breakdown products derived from bronchial wall cartilage. Nevertheless, such a limitation may not affect the validity of the test as a measure of emphysematous lung injury because damage to the central airways may involve the same processes that occur at the alveolar level (32). Moreover, macrophages containing ingested fragments of alveolar wall elastic fibers may be present in sputum from a deep cough, thereby facilitating assessment of peripheral lung injury (33).

Clinical Trial of the Use of Sputum Desmosine and Isodesmosine as Therapeutic Markers

Hyaluronan aerosol in experimental studies in animals has been shown to: 1) decrease the severity of emphysema induced by elastase enzymes (34,35); 2) block elastase degradation of elastin fibers in vitro (36); 3) decrease the pulmonary emphysema induced in mice exposed to tobacco smoke over a 6-month period (37); 4) improve tolerability of hypertonic saline as a therapy in cystic fibrosis (38); 5) reduce lung inflammation in a cystic fibrosis model in mice (39); It is also recognized that hyaluronan content of lung tissue in COPD is reduced (40,41) and exposure to tobacco smoke degrades hyaluronan in vivo and in vivo (42,43).

With this background, efforts continue to develop hyaluronan aerosol as a potential therapy for chronic obstructive pulmonary disease (COPD) related to alpha-1 antitrypsin deficiency (AATD) and not related to AATD. As part of that development this clinical safety trial was conducted in 11 COPD patients, 8 of whom received hyaluronan and 3 received placebo. The study protocol was conducted under IND number 70299. The primary aim of the study was to establish safety and to evaluate biomarker activity with respect to body and lung elastin degradation.

Eleven patients were recruited from 2 Centers: 9 patients from Research Associates in Tucson Ariz. and 2 patients from St. Luke's-Roosevelt Hospital Pulmonary Disease Center in New York. All patients fulfilled the diagnostic criteria of COPD with GOLD grades 2 and 3 with moderate airway obstruction (FEV₁ above 40% of predicted) and at least a ten pack-year history of cigarette smoking. No patients were actively smoking at the time of recruitment and smoking cessation had occurred over one year prior to the study in all patients. Six patients were male and 4 patients female. All were Caucasian. Three patients received placebo. Eight patients received hyaluronan CTX-100. Adverse events were mild to moderate and recurred with a greater frequency of 67% in the placebo group.

Each patient self-administered 0.01% hyaluronan from a vialed volume of 3 ml by aerosol from a compressor-delivered Pari nebulizer, morning and night for 14 days. The primary endpoints to assess safety were adverse events, oxygen saturation, spirometry, lung volumes, physical examination, vital signs, electrocardiogram and laboratory evaluations (CBC, serum chemistries and urinalysis). Additionally, desmosine and isodesmosine concentrations in sputum, plasma and urine were measured to assess a possible effect on elastin degradation (44, 45)

There was no significant effect of 2 weeks of administration of CTX-100 on spirometric indices, routine laboratory procedures, and EKG. However, the measurements of desmosine and isodesmosoine showed a statistically significant reduction of levels of desmosine and isodesmsoine in all 6 patients who provided samples of sputum and received hyaluronan aerosol (FIG. 1). There was also a significant reduction in blood and urine levels of desmosine and isodesmosine.

### CONCLUSIONS

Emphysema is a chronic, debilitating disease that lacks satisfactory treatment. Cessation of smoking slows the progression of the disease, but does not prevent further lung damage. Physicians generally prescribe antibiotics to suppress infections, bronchodilators to relax and dilate constricted bronchi, and systemic steroids such as prednisone to reduce inflammation. In the final stages, supplementary oxygen eases the functional burden of the lungs

As new treatments for pulmonary emphysema begin to emerge, the need for a rapid assessment of their efficacy will grow in importance. Currently, the only recognized endpoints to determine therapeutic efficacy are pulmonary function studies, which may take years to detect a significant effect. The delay is due to the fact that pulmonary emphysema progresses at a relatively slow rate and available methods for measuring loss of lung function are not particularly sensitive. Determination of lung density using HRCT has been proposed as a more sensitive alternative to lung function studies, but this methodology may also require an extended period of time to determine any treatment effect. These limitations have hindered evaluation of new therapeutic agents for this disease, primarily because the commercial sector is unwilling to invest the substantial time and funds required to determine if such treatments are worth bringing to the marketplace.

In the case of our own laboratory, the need for a sensitive measurement of lung elastic fiber injury is immediate. We have developed a novel approach to treating pulmonary emphysema involving the use of aerosolized hyaluronan to directly protect elastic fibers from injury (34, 35). However, the advancement of this potential treatment will require the availability of a short-term endpoint to assess its efficacy. Indeed, the development of a simple, reliable test to assess changes in the progression of emphysematous lung injury would have important consequences not only for our work, but for this entire field of research as well.

### REFERENCES

1. Suki B, Lutchen K R, Ingenito E P: On the progressive nature of emphysema: roles of proteases, inflammation, and mechanical forces. Am J Respir Crit Care Med. 2003; 168: 516-521.
2. Shapiro S D: Proteinases in chronic obstructive pulmonary disease. Biochem Soc Trans. 2002; 30: 98-102.
3. Barnes, P J. Chronic obstructive pulmonary disease. NEJM 343:269-278, 2000.
4. Mannino D M, Homa D M, Akinbami L J, Ford E S, Redd S C. Chronic obstructive pulmonary disease surveillance—United States, 1971-2000. MMWR Surveill Summ. 2002 Aug. 2; 51(6):1-16.
5. Gross P, Babyak M A, Tolker E, & Kaschak, M. Enzymatically produced pulmonary emphysema: a preliminary report. J Occup Med 1964; 6:481-484.
6. Laurell C-B, Eriksson S. The electrophoretic alpha1-globulin pattern of serum in alpha1-antitrypsin deficiency. Scand J Clin Lab Invest 1963; 15:132-140.
7. Alpha-1-antitrypsin deficiency registry study group: survival and FEV1 decline in individuals with severe deficiency of α₁-antitrypsin. Am J Resp Crit Care Med 158:49-59, 1998.
8. Seersholm N, Wencker M, Banik N et al. Does α₁-antitrypsin augmentation therapy slow the annual decline in FEV1 in patients with severe hereditary α₁-antitrypsin deficiency? Eur Respir J 10:2260-63, 1997.
9. Nakano Y, Muller N L, King G G, Niimi A, Kalloger S E, Mishima M, Pare P D. Quantitative assessment of airway remodeling using high-resolution CT. Chest 2002; 122:271 S-5S.
10. Dirksen A, Friis M, Olesen K P, Skovgaard L T, Sorensen K. Progress of emphysema in severe á1-antitrypsin deficiency as assessed by annual CT. Acta Radiol 38:826-32, 1997.
11. Biernacki W, Ryan M, MacNee W, Flenley D C. Can quantitative CT scanning detect the progression of emphysema? Am Rev Respir Dis 139:A120, 1989.
12. Churg A, Wang R D, Hsin T, Wang X, Changshi X, Wright J L. Tumor necrosis factor-α drives 70% of cigarette smoke-induced emphysema in the mouse. Am J Respir Crit Care Med 2004; 170:492-498.
13. Barnes P J. Mediators of chronic obstructive pulmonary disease. Pharmacol Rev 2004 56:515-548.
14. Mecham R P. Elastin synthesis and fiber assembly. Ann NY Acad Sci 1991; 624:137-146.
15. Fiorenza D, Viglio S, Lupi A, Baccheschi J, Tinelli C, Trisolini R, Iadarola R, Luisetti M, Snider G L. Urinary desmosine excretion in acute exacerbations of COPD: a preliminary report. Respir Med 2002; 96:110-4.
16. Viglio S, Iadorola P, Lupi A, Trisolini R, Tinelli C, Balbi B, Grassi V, Worlitzsch D, Doring G, Meloni F, Meyer K C, Dowson L, Hill S L, Stockley R A, Luisetti M. MEKC of desmosine and isodesmosine in urine of chronic destructive lung disease patients. Eur Respir J 200; 15:1039-45.
17. Starcher B, Green M, Scott M. Measurement of urinary desmosine as an indicator of acute pulmonary disease. Respiration 1995; 62:252-7.
Idell S, Thrall R S, Maunder R, Martin T R, McLarty J, Scott M, Sarcher B C. Bronchoalveolar lavage desmosine in bleomycin-induced lung injury in marmosets and patients with adult respiratory distress syndrome. Exp Lung Res 1989; 15:739-53.
19. Bruce M C, Schuyler M, Martin R J, Starcher B C, Tomashefski J F Jr, Wedig K E. Risk factors for the degradation of lung elastic fibers in the ventilated neonate. Implications for impaired lung development in bronchopulmonary dysplasia. Am Rev Respir Dis. 1992; 146:204-12.
20. Volkovysskaia V B, Nepomniashchikh. Staining elastic fibers in sputum. Lab Delo 1969; 7:415-7.
21. Shlaes D M, Lederman M, Chmielewski R, Tweardy D, Wolinsky E. Elastin fibers in the sputum of patients with necrotizing pneumonia. Chest 1983; 83:885-
22. Shlaes D M, Lederman M M, Chmielewski R, Tweardy D, Krause G, Saffai C. Sputum elastic fibers and the diagnosis of necrotizing pneumonia. Chest 1984; 85:763-6.
23. Cantor J O, Shteyngart B. Measurement of elastic fiber breakdown products in sputum. U.S. patent application Ser. No. 10/253,756, 2002.
24. Viglio S, Zanaboni G, Luisetti M, Trisolini R, Grimm R, Cetta G, Iadarola P. Micellar electrokinetic chromatography for the determination of urinary desmosine and isodesmosine in patients affected by chronic obstructive pulmonary disease. J Chromatogr B Biomed Sc Appl 1998; 714:87-98.
25. Stone P J, Bryan-Rhadfi J, Lucey E C, Ciccolella D E, Crombie G, Faris B, Snider G L, Franzblau C. Measurement of urinary desmosine by isotope dilution and high performance liquid chromatography. Correlation between elastase-induced air-space enlargement in the hamster and elevation of urinary desmosine. Am Rev Respir Dis 144:284-290, 1991.
26. Soskel N T. High-performance liquid chromatographic quantitation of desmosine plus isodesmosine in elastin and whole tissue hydrolysates. Anal Biochem 1987; 160:98-104.
29. Ma S, Lieberman S, Turino G M, Lin Y Y. The detection and quantitation of free desmosine and isodesmosine in human urine and their peptide-bound forms in sputum. Proc Natl Acad Sci USA 100:12,941-12,943.
30. Boschetto P, Quintavalle S, Zeni E, Leprotti S, Potena A, Ballerin L, Papi A, Palladini G, Luisetti M, Annovazzi L, Iadorola P, De Rosa E, Fabbri L M, Mapp C E. Association between markers of emphysema and more severe chronic obstructive pulmonary disease. Thorax 2006; 61:1037-1042.
31. Beeh K M, Beier J, Kornmann O, Mander A, Buhl R. Long-term repeatability of induced sputum cells and inflammatory markers in stable, moderately severe COPD. Chest 2003; 123:778-83.
32. Hill A, Gompertz S, Stockley R. Factors influencing airway inflammation in chronic obstructive pulmonary disease. Thorax 2000; 55:970-7.
33. Senior R M, Griffin G L, Mecham R P. Chemotactic activity of elastin-derived peptides. J Clin Invest 1980; 66:859-62.
34. Cantor J and Turino G M: Modulation of air-space enlargement in elastase-induced emphysema by intratracheal instillment of hyaluronidase and hyaluronan. Exper Lung Res 1995, 21(3):423-36.
35. Cantor J O, Cerreta Joseph M, Armand G and Turino G M: Aerosolized hyaluronan decreases alveolar injury induced by human neutrophil elastase. Proceedings Soc Exper Biol & Med 1998, 217:471-75.
36. Cantor J O, Shteyngart B, Cerreta J M, Liu M, Armand G and Turino G M: The effect of hyaluronan on elastic fiber injury in vitro and elastase-induced airspace enlargement in vivo. Proc Soc for Exp Biol & Med 2000, 225:65-71.
37. Cantor J O, Cerreta J M, Ochoa M, Ma S, Chow T, Grunig G and Turino G M: Aerosolized hyaluronan limits airspace enlargement in a mouse model of cigarette smoke-induced pulmonary emphysema. Accepted for publication in Experimental Lung Research (2004).
38. Furnari M L, Termini L, Traverso G, Barrale S, Bonaccorso M R, Damiani G, L Piparo C and Collura M: Nebulized hypertonic saline containing hyaluronan improves tolerance in patients with cystic firbrosis and lung disease compared with nebulized hypertonic saline alone: a prospective, randomized, couble-blind controlled study. Therapeutic Advances in Respiratory Disease 2012, 10:1177:1-8.
39. Gavina M, Luciani A, Villella V R, Esposito S, Ferrari E, Bressani I, Casale A, Bruscia E, Maiuri L and Raia V: Nebulized Hyaluronan ameliorates lung inflammation in Cystic Fibrosis mice. Pediatric Pulmonology 2012, doi: 10.1002/ppul.22637.
40. Konno K, Arai H, Motomiya M, Nagai H, Ito M, Sato H and Satoh K: A biochemical study on glycosaminoglycans (mucopolysaccharides) in emphysematous and in aged lungs. Am Rev Respir Dis 1982, 126:797-801.
41. Klagas I, Goulet S, Karakiulakis G, Zhong J, Baraket M, Black J L, Papakonstansinou E and Roth M: Decreased hyaluronan in airway smooth muscle cells from patients with asthma and COPD. Eur Respir J 2009, 34:616-28.
42. McDevitt et al McDevitt C A, Beck G J, Ciunga M J and O'Brien J: Cigarette smoke degrades hyaluronan. Lung 1989, 167:237-45.
43. Dabrowski R and Maslinski C: Collagen, glycosaminoglycans and histamine in lungs of Guinea pigs exposed chronically to cigarette smoke. Bull Acad Pol Des Sci 1975, 23:125-28.
44. Ma S, Lieberman S, Turino G M and Lin Y Y: The detection and quantitation of free desmosine and isodesmosine in human urine and their peptide-bound forms in sputum. Proceed Natl Acad Sci Oct. 28, 2003, 100(22):12941-43.
45. Ma S, Lin Y Y and Turino G M: Measurements of Desmosine and Isodesmosine by Mass Spectrometry in COPD. CHEST 2007, 131:1363

### SUMMARY OF INVENTION

The current invention comprises a method of adjusting the dosage of a drug that prevents elastic fiber damage by employing a feedback loop consisting of measuring the levels of the unique elastic fiber breakdown products, desmosine and isodesmosine, primarily in sputum. Neither of these molecules is a natural product of body metabolism because they only appear in sputum in pathological conditions involving the lung. Furthermore, desmosine and isodesmosine are largely bound to peptides in body fluids, and require the use of acid hydrolysis to separate them for measurement, a process which obviates their consideration as a natural body product.

The sputum level of desmosine and isodesmosine thus reflects damage to lung elastic fibers and may therefore serve as a biomarker for the effectiveness of aerosolized hyaluronan and other drugs that prevent elastic fiber damage in the treatment of respiratory disorders that involve elastic fiber breakdown, such as pulmonary emphysema, chronic bronchitis, asthma, pulmonary edema, acute respiratory distress syndrome (adult and neonatal), bronchopulmonary dysplasia, interstitial pulmonary fibrosis, cystic fibrosis, pneumonia, and pulmonary atelectasis.

This method may be used to adjust the dosage of any drug that prevents elastic fiber breakdown, including hyaluronan, alpha-1-antitrypsin, various other enzyme inhibitors, and antioxidants. In the preferred embodiment, measurement of desmosine and isodesmosine in either sputum, blood, urine, or saliva samples may be used to adjust the dosage of hyaluronan.

Administration of hyaluronan may be performed by aerosol, which can be generated by a nebulizer, or by instillation (Cantor, U.S. Pat. No. 5,633,003 and U.S. Pat. No. 6,391,861). The hyaluronan may be administered alone, or with a carrier such as saline solution, DMSO, an alcohol or water. It may be isolated from a natural source such as a bovine or rooster. The effective daily amount of hyaluronan is from about 10 micrograms/kg to about 1 mg/kg of body weight.

Measurement of the elastic fiber breakdown products, desmosine and isodesmosine, in sputum may be used to monitor the efficacy of hyaluronan treatment (Cantor and Shteyngart, U.S. Pat. No. 7,166,437). Sputum levels of desmosine and isodesmosine are associated with lung injury resulting from degradation of lung elastic fibers. This process may occur in emphysema and other inflammatory diseases of the lung where excess amounts of elastase are secreted by inflammatory cells. The methods for measuring elastin breakdown products in certain tissue fluids (i.e. blood, urine, bronchoalveolar lavage fluid) are well known to the art but have not been previously applied to sputum samples. The use of sputum has several advantages over other fluids, including greater specificity and ease of procurement.

### BRIEF DESCRIPTION OF FIGURES

FIG. 1:

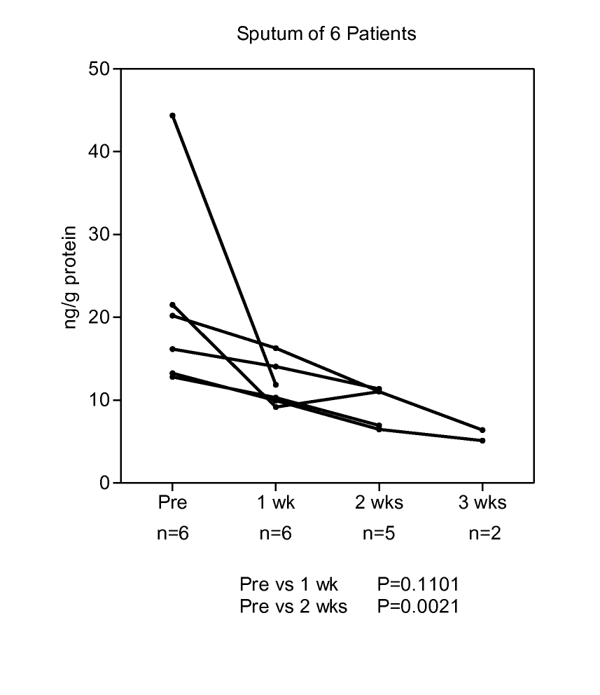

Reductions in levels of desmosine and isodesmosine in sputum during and after 2 weeks of hyaluronan aerosol administration.

### DETAILED DESCRIPTION OF INVENTION

The current invention comprises a method of adjusting the dosage of a drug that prevents elastic fiber damage by employing a feedback loop consisting of measuring the levels of the unique elastic fiber breakdown products, desmosine and isodesmosine, primarily in sputum. Neither of these molecules is a natural product of body metabolism because they only appear in sputum in pathological conditions involving the lung. Furthermore, desmosine and isodesmosine are largely bound to peptides in body fluids, and require the use of acid hydrolysis to separate them for measurement, a process which obviates their consideration as a natural body product.

In subjects with a disease that involves breakdown of elastic fibers, the desired goal of treatment is to reduce the breakdown of these fibers such that the destruction of tissue that depends on the elastic fibers for mechanical support is also decreased. In the case of COPD, the loss of elastic fibers results in dilatation and rupture of alveolar walls, thereby reducing lung surface area and impairing gas exchange. Prior to the applicants' studies enumerated above, there has been no effective way of adjusting the dosage of a drug that prevents elastic fiber breakdown because the available tests for such damage did not have sufficient sensitivity and/or specificity to permit real-time assessment of therapeutic efficacy. The methods described in the current invention provide the means of increasing the effectiveness of a drug that prevents elastic fiber breakdown by customizing dosages to the individual response of the patient being treated with the drug.

Drugs that can be used to prevent elastic fiber breakdown include elastase inhibitors (e.g. alpha-1 antiproteinase), antioxidants, and compounds that bind to elastic fibers and prevent their breakdown by elastases and/or oxidants, such as hyaluronan. As described above, the applicants have tested the therapeutic efficacy of aerosolized hyaluronan by measuring the levels of desmosine and isodesmosine in sputum, blood, and urine. These studies indicate that administration of aerosolized hyaluronan results in a statistically significant decline in sputum, blood, and urine levels of desmosine and isodesmosine in the first two weeks of treatment. This information provides a new and important feedback loop for treatment of diseases that involve elastic fiber breakdown.

In the one embodiment of the current invention, the measurement of sputum desmosine and isodesmosine would be used to adjust the dosage of aerosolized hyaluronan, which prevents elastic fiber injury in the lung. The sputum level of desmosine and isodesmosine reflects damage to lung elastic fibers and may therefore serve as a biomarker for the effectiveness of aerosolized hyaluronan in the treatment of respiratory disorders that involve elastic fiber breakdown, such as pulmonary emphysema, chronic bronchitis, asthma, pulmonary edema, acute respiratory distress syndrome (adult and neonatal), bronchopulmonary dysplasia, interstitial pulmonary fibrosis, cystic fibrosis, pneumonia, and pulmonary atelectasis.

Administration of hyaluronan may be performed by aerosol, which can be generated by a nebulizer, or by instillation (Cantor, U.S. Pat. No. 5,633,003 and U.S. Pat. No. 6,391,861). The hyaluronan may be administered alone, or with a carrier such as saline solution, DMSO, an alcohol or water. It may be isolated from a natural source such as a bovine or rooster.

The effective daily amount of hyaluronan administered intratracheally may vary from about 10 micrograms/kg to about 1 mg/kg of body weight of the human being treated. Preferably, the daily amount is from about 10 micrograms/kg to about 100 micrograms/kg, for example about 50 micorgrams/kg body weight of the human being treated (daily). The intratracheal hyaluronan may be administered in any other of the methods well known to those skilled in the art. For example, the hyaluronan may be administered in the form of an aerosol or may be administered by instillation. If administered in the form of an aerosol, a nebulizer is used to produce hyaluronan in aerosol form (See for example U.S. Pat. No. 4,649,911 and U.S. Pat. No. 4,119,096).

Typically, the hyaluronan is administered in a pharmaceutically acceptable carrier, such examples include saline solution, DMSO, an alcohol or water. Such carriers are well known in the art, and the specific carriers employed may be varied depending upon factors such as size of the subject being treated, treatment dose and the like.

Further, the time over which the hyaluronan is administered may vary as is well known in the art to achieve the desired results. For example, the hyaluronan may be administered as an aerosol from about 10 minutes to about 1 hour per treatment regimen, three times daily or until the desired daily dosage is fully administered.

In addition, forms of hyaluronan may be derived from bovine sources, rooster comb, human umbilical cord, or streptoccus zoepidicus (See U.S. Pat. No. 4,780,414 and U.S. Pat. No. 4,784,990). All forms of hyaluronan, regardless of source, would follow a treatment similar to that described above.

Measurement of the elastic fiber breakdown products, desmosine and isodesmosine, in sputum may be used to monitor the efficacy of hyaluronan treatment (Cantor and Shteyngart, U.S. Pat. No. 7,166,437). Sputum levels of desmosine and isodesmosine are associated with lung injury resulting from degradation of lung elastic fibers. This process may occur in emphysema and other inflammatory diseases of the lung where excess amounts of elastase are secreted by inflammatory cells. The methods for measuring elastin breakdown products in certain tissue fluids (i.e. blood, urine, bronchoalveolar lavage fluid) are well known to the art but have not been previously applied to sputum samples. The use of sputum has several advantages over other fluids, including greater specificity and ease of procurement. The following protocol incorporates the preferred embodiment of the subject invention.

Sputum samples collected from patients with emphysema and other inflammatory lung diseases are chemically degraded to separate the component amino acids of elastic fibers and other proteins. This procedure may involve hydrolyzing the sample with an acid such as HCl or a base such as NaOH, according to procedures well known to the art. The resulting hydrolysate is then measured for total elastin-specific amino acids, desmosine and isodesmosine, using a variety of procedures, including liquid chromatography or immunoassay, ultraviolet spectroscopy, fluorescence spectroscopy, mass spectrometry, or other spectroscopic techniques well-known to the art.

Desmosine and isodesmosine content of the sputum sample is then normalized to one or more parameters, such as total protein, albumin, or free amino acids, according to procedures well known to the art. The final value is expressed as total desmosine/isodesmosine per unit volume, protein, albumin, amino acids or other relevant parameter. Results obtained from the same patient over time may be used to determine variations in the amount of lung elastic fiber injury.

The invention may be used to determine whether a patient requires treatment with a drug that prevents elastic fiber breakdown. The protocol for this embodiment of the invention would involve the following steps: a) measuring the level of desmosine and isodesmosine in sputum; b) comparing the sputum level of desmosine and isodesmosine to the upper limit of normal for sputum desmosine and isodesmosine level, and c) administering a drug that prevents elastic fiber breakdown if the sputum level of desmosine and isodesmosine is greater than the upper limit of normal for sputum desmosine and isodesmosine.

The invention may also be used to adjust the dosage of a drug that prevents elastic fiber breakdown in a subject who has previously been administered an initial dosage of a drug that prevents elastic fiber breakdown. The protocol for this embodiment of the invention would involve the following steps: a) measuring the level of the elastin breakdown products, desmosine and isodesmosine, in sputum; b) comparing the sputum level of desmosine and isodesmosine to the upper limit of normal for sputum desmosine and isodesmosine, and c) administering an adjusted dosage of a drug that prevents elastic fiber breakdown wherein the adjusted dosage is greater than the initial dosage if the sputum level of desmosine and isodesmosine is greater than the upper limit of normal for sputum desmosine and isodesmosine.

Examples of the use of the invention to titrate treatment with aerosolized hyaluronan are given below:

1) A female patient with pulmonary emphysema is found to have an abnormally high level of sputum desmosine and isodesmosine (20 nanograms/gram protein compared to a normal level of less than 5 nanograms/g protein), and begins treatment with a dose of 0.1% hyaluronan that is inhaled for 20 minutes once daily in the morning. After 1 month, the sputum desmosine and isodesmosine level is measured and found to be 10 nanograms/g of protein in the sputum, which is more than twice the upper limit of normal. The patient is then switched to a twice daily inhalation for 20 minutes in the morning and evening. Two weeks later, the patient's sputum desmosine and isodesmosine level has fallen to 1 nanogram/g of protein, which is within the normal range. Her treatment is therefore left unchanged and sputum desmosine and isodesmsoine levels are measured at monthly intervals to ensure that the drug remains effective in preventing elastic fiber breakdown.

2) A male patient with a history of smoking is tested for sputum desmosine and isodesmosine levels. He is found to have a mildly elevated level of 6 nanograms/g protein, and is placed on low dose hyaluronan, involving a single daily 20 minute inhalation of 0.05% hyaluronan. Two weeks later, his sputum desmosine and isodesmosine level is 1 nanogram/g protein, which is within the normal range. Based on this finding, his treatment is lowered to a 20 minute inhalation of hyaluronan every other day. Two weeks later, his sputum desmosine and isodesmsoine level has risen to 6 nanograms/g protein, which is above the upper limit of normal. His treatment regimen is therefore returned to a 20 minute inhalation every day. Two weeks later, his sputum desmosine and isodesmosine level has fallen to 1 nanogram/g protein. His treatment needs no further modification, and sputum desmosine and isodesmosine levels are measured at monthly intervals to ensure that the drug remains effective in preventing elastic fiber breakdown.

The advantages of this new approach to treatment include:

a. Rapid determination of the effectiveness of treatment.

b. Adjustment of the dosage of the drug to improve its effectiveness.

c. Reduced risk of potential side-effects, such as pulmonary inflammation, due to unnecessarily high levels of the drug in the patient's lungs.

d. Improved patient compliance due to demonstration of the drug's effectiveness.

The novel aspects of the invention include:

a) Linkage of sputum desmosine and isodesmosine to treatment regimens

b) Determination of the upper limit of normal for sputum desmosine and isodesmosine.

c) Proof of concept that the effectiveness of a drug that prevents elastic fiber breakdown may be monitored by measuring sputum levels of desmosine and isodesmosine.

d) Reduction in the risk factors associated with potential side-effects that may occur if the drug is maintained at a higher than needed dosage.

e) Rapid screening of drugs that prevent elastic fiber breakdown for their effectiveness, thereby reducing financial burden of extended clinical trials with less sensitive endpoints.

None of these features could be anticipated by one skilled in the art for the following reasons:

a) Clinical studies were needed to confirm that sputum desmosine and isodesmosine is a useful biomarker for the effectiveness of drugs that prevent elastic fiber breakdown.

b) Clinical trials were needed to show the rapid response of sputum levels of desmosine and isodesmosine to drug treatment, which validates the use of these levels as a real-time biomarker for adjusting drug dosage.

c) The range of normal values for sputum desmosine and isodesmosine levels has only recently been determined by the applicants. This information was previously unavailable to one skilled in the art. A large variance in sputum desmosine and isodesmosine levels in a population without COPD would have invalidated the use of this biomarker for the purpose of adjusting the dosage of drugs that prevent elastic fiber breakdown.

In an alternative embodiment of the invention, the level of desmosine and isodesmosine in blood and or urine may be used to monitor the effectiveness of a drug that prevents elastic fiber breakdown. The protocol for using blood or urine levels of desmosine and isodesmosine to adjust drug levels would be similar to that described above for sputum desmosine and isodesmosine. Furthermore, sputum desmosine and isodesmsoine levels may be used in conjunction with blood or urine levels of desmosine and isodesmosine to adjust the dosage of a drug that prevents elastic fiber breakdown. For blood and urine measurements, the ratio of free to total desmosine and isodesmosine may be used, as well as the ratio of free to peptide-bound desmosine and isodesmosine. Studies by the applicants indicate an upper limit of normal for the ratio of free to total desmosine in the range of 0.2 to 0.4, and an upper limit of normal for free to peptide-bound desmosine and isodesmosine of approximately 0.3 to 0.5.

In another embodiment of the invention, sputum, blood, or urine levels of desmosine and isodesmosine (or blood and urine ratios of free to total or free to peptide-bound desmosine and isodesmosine) are used to adjust the dosage of a chemical derivative of hyaluronan that prevents elastic fiber breakdown.

In another embodiment of the invention, sputum, blood, or urine levels of desmosine (or blood and urine ratios of free to total or free to peptide-bound desmosine and isodesmosine) are used to adjust the dosage of an elastase inhibitor (e.g. alpha-1 antiproteinase) that prevents elastic fiber breakdown.

What is claimed is:

1. A method for adjusting the dosage of a drug that prevents elastic fiber breakdown in a subject who has previously been administered an initial dosage of a drug that prevents elastic fiber breakdown, comprising: a) measuring the level of the elastin breakdown products, desmosine and isodesmosine, in sputum; b) comparing the sputum level of desmosine and isodesmosine to the upper limit of normal for sputum desmosine and isodesmosine, and c) administering an adjusted dosage of a drug that prevents elastic fiber breakdown wherein the adjusted dosage is greater than the initial dosage if the sputum level of desmosine and isodesmosine is greater than the upper limit of normal for sputum desmosine and isodesmosine.

2. A method of administering a drug that prevents elastic fiber breakdown to a subject having a disease that involves elastic fiber breakdown comprising: a) measuring the level of desmosine and isodesmosine in sputum; b) comparing the sputum level of desmosine and isodesmosine to the upper limit of normal for sputum desmosine and isodesmosine level, and c) administering a drug that prevents elastic fiber breakdown if the sputum level of desmosine and isodesmosine is greater than the upper limit of normal for sputum desmosine and isodesmosine.

3. A method of treating a subject with a disease involving elastic fiber breakdown who has previously been administered an initial dosage of a drug that prevents elastic fiber breakdown comprising: a) measuring the level of desmosine and isodesmosine in the sputum of the subject; b) comparing the sputum level of desmosine and isodesmosine to the upper limit of normal for sputum desmosine and isodesmosine; and c) administering an adjusted dosage of a drug that prevents elastic fiber breakdown if the sputum level of desmosine and isodesmosine is greater than the upper limit of normal for sputum desmosine and isodesmosine.

4. The method of any of claims 1-3, wherein the disease involving elastic fiber breakdown is selected from the group consisting of pulmonary emphysema, chronic bronchitis, asthma, pulmonary edema, acute respiratory distress syndrome (adult and neonatal), bronchopulmonary dysplasia, interstitial pulmonary fibrosis, cystic fibrosis, pneumonia, and pulmonary atelectasis.

5. The method of any of claims 1-3, wherein the drug that prevents elastic fiber breakdown is hyaluronan, or a chemical derivative of hyaluronan.

6. The method of any of claims 1-3, wherein the drug that prevents elastic fiber breakdown is an elastase inhibitor, such as alpha-1 antitrypsin.

7. The method of claim 3 or 4, wherein administering an increased dosage of the drug that prevents elastic fiber breakdown produces a normal weekly level of sputum desmosine and isodesmsoine in the subject.

8. The method of any of claims 1-3, further comprising the step of determining the upper limit of normal for the subject prior to step (b).

9. The method of any of claims 1-3, wherein the upper limit of normal for sputum desmosine and isodesmosine is less than 5 nanograms per gram protein.

10. The method of any of claims 1-3, wherein the level of desmosine and isodesmosine in blood or urine is used as an alternative to the sputum level of desmosine and isodesmosine.

11. The method of any of claims 1-3, wherein the ratio of free to total desmosine and isodesmosine in blood and urine is used as an alternative to the sputum level of desmosine and isodesmosine.

12. The method of any of claims 1-3, wherein the ratio of free to peptide-bound desmosine and isodesmosine in blood and urine is used as an alternative to the sputum level of desmosine and isodesmosine.

13. The method of any of claims 1-3, wherein the upper limit of normal for the ratio of free to total desmosine and isodesmosine is in the range of 0.2 to 0.4.

14. The method of any of claims 1-3, wherein the upper limit of normal for the ratio of free to peptide-bound desmosine and isodesmosine is in the range of 0.3 to 0.5.
